(19) United States
(12) Patent Application Publication
(10) Pub. No.: US 2003/0199816 A1
(43) Pub. Date: Oct. 23, 2003

(54) Pre-loaded multi-chamber syringe

(76) Inventor: Scott Ramming, Weaverville, NC (US)

Correspondence Address: David M. Carter, Carter & Schnedler, P.A., 56 Central Avenue, Suite 101, P.O. Box 2985, Asheville, NC, 28802, US

(21) Appl. No.: 10/124,916

(22) Filed: Apr. 17, 2002

(51) Int. Cl. A61M 37/00

(52) U.S. Cl. 604/89; 604/191

(57) ABSTRACT

There is provided a pre-loaded multi-chamber syringe including an elongated hollow tube having first and second ends. The first end of the tube receives a needle support and the second end of the tube receives a plunger assembly. A moveable gasket is received between the one end of the plunger assembly and the first end of the tube thereby forming first and second chambers. Each chamber receives a liquid. A hollow needle extends from the needle assembly initially into the first chamber. When the plunger is depressed, the liquid from the first chamber is expelled through the hollow needle and the gasket moves to come in contact with the hollow needle wherein the hollow needle penetrates the gasket and thus extends into the second chamber so that the liquid from the second chamber is then expelled through the hollow needle.

### FIELD OF THE INVENTION

The present invention relates to an apparatus and method for the rapid administration of medicine to a patient. More particularly, it relates to the rapid administration of medicine to a patient through using a syringe.

### BACKGROUND OF THE INVENTION

This invention relates to an apparatus and method for the rapid administration of medicine to a patient. More particularly, it relates to the rapid administration of medicine to a patient through using a syringe. Medical patients are often catheterized with an intravenous (IV) line having an injection port so that medicines may be promptly injected into the patient through the use of a syringe. It is often necessary to insert saline or some other solution into the IV line following the administration of a medicine. For example, there is a need to quickly administer a substantial amount of saline immediately following the use of adenosine, a medicine which is ineffective if it is not delivered to the central circulation in a very rapid manner.

In addition, it is often appropriate to clear IV catheters with saline after the use of most medications. Currently, medicines are inserted into an IV catheter through the use of first syringe and afterwards, saline is inserted to the catheter through the use of a second syringe. In the case of the use of adenosine it can be problematic for the health care worker to quickly administer the saline after the administration of the adenosine because of the need to use two separate syringes.

### OBJECTS OF THE INVENTION

It is therefore one object of this invention to provide a device and method which enables the rapid and efficient administration of saline or other solutions to IV catheters, subsequent to the administration of medication.

It is another object to enable the administration of saline or other fluids to an IV line subsequent to the administration of a medicine without need to use a second syringe.

### SUMMARY OF THE INVENTION

In accordance with one form of this invention there is provided a pre-loaded multi-chambered syringe including an elongated hollow tubular barrel, having first and second open ends and an inner wall. A needle assembly is attached to the first end of the barrel. A plunger assembly is slidably received inside the barrel through the second end. The plunger assembly has a first gasket engaging the inner wall in substantially an interference fit. A second gasket engages the inner wall also in a substantially interference fit. The second gasket is slidably received inside the barrel and is located between the first gasket and the first end, so that a first chamber is formed between the second gasket and the first end, and a second chamber is formed between the second gasket and the first gasket. The first chamber is substantially filled with a first material, preferably a liquid medicine. The second chamber is substantially filled with a liquid, preferably saline solution, so that the movement of the first gasket towards the first end causes the second gasket to slide towards the first end, thereby expelling the first material through the needle assembly. The second gasket together with the portion of the wall interfacing with the second gasket maintains a substantially liquid impervious seal between the first chamber and the second chamber. The seal between the first and second chamber is maintained while the second gasket slides toward the first end of the barrel. The needle assembly is adapted to penetrate the second seal so that an amount of liquid in the second chamber can be expelled through the needle assembly after the first material has been expelled.

In accordance with another form of this invention there is provided a multi-chambered syringe including an elongated hollow tube having an inside surface and first and second open ends. The first end receives a needle support. The second end receives a plunger assembly. A portion of the plunger assembly forms a seal with the inside surface of the tube. A moveable gasket is located between the plunger assembly and the first end. A first chamber is formed between the gasket and the first end. A second chamber is formed between the gasket and the plunger. A hollow needle is attached to the needle support and extends into the first chamber. The first chamber is substantially filled with a first liquid. The second chamber is substantially filled with a second liquid. When the plunger is depressed the gasket will move toward the first opening for a first distance, expelling the first liquid through the needle and when the plunger is further depressed the gasket will move towards the first opening a second distance and the needle will penetrate through the gasket and into the second chamber, thereby expelling the second liquid through the needle.

In accordance with another form of this invention there is provided a method for expelling at least two separate liquids from a multi-chamber syringe. The syringe includes an elongated hollow tube having an inside surface and first and second open ends. The first end receives a needle support. The second end receives a plunger assembly. A portion of the plunger assembly forms a seal with the inside surface of the tube. A moveable gasket is located between the plunger assembly and the first end. A first chamber is formed between the gasket and the first end, and a second chamber is formed between the gasket and the plunger assembly. A hollow needle is attached to the needle support and extends into the first chamber. The first chamber is substantially filled with a first liquid and the second chamber is substantially filled with a second liquid. The method includes the steps of depressing the plunger a first distance, moving the gasket towards the first end, expelling an amount of the first liquid through the needle, depressing the plunger a second distance, penetrating the gasket with the needle, extending the needle into the second chamber and expelling an amount of the second liquid through the needle.

### BRIEF DESCRIPTION OF THE DRAWINGS

The subject matter which is regarded as the invention as set forth in the appended claims, the invention itself however, together with further objects and advantages thereof may be better understood in reference to the accompanying drawings in which:

FIG. 1 is a pictorial view of the multi-chamber syringe of the present invention showing the plunger in an undepressed or partially depressed position.

FIG. 2 is a pictorial view of the syringe of FIG. 1 however, showing the plunger in a more fully depressed position.

FIG. 3 is pictorial view of the needle assembly, of FIGS. 1 and 2, showing more details of the needle assembly.

### DESCRIPTION OF THE PREFERRED EMBODIMENTS

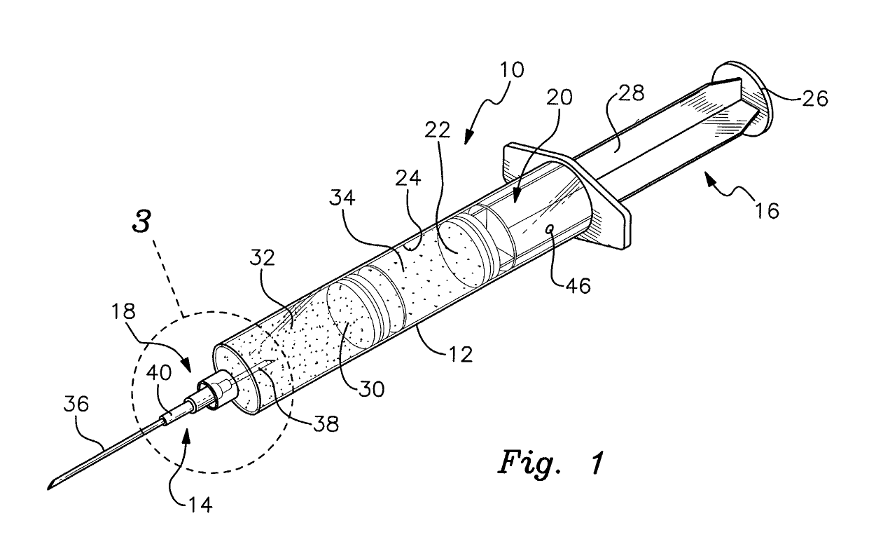

Referring now, more particularly to FIG. 1, there is provided multi-chambered syringe 10 including elongated hollow barrel or tube 12, a needle assembly 14 and a plunger assembly 16. Tube 12 has a first open end 18 for receiving needle assembly 14 and a second open end 20 for receiving plunger assembly 16. Plunger assembly 16 is slidably received within tube 12. Plunger assembly 16 may be a standard syringe plunger including a gasket or seal 22 which fits snugly against the inside curved wall 24 of tube 12 in substantially an interference fit. Seal 22 preferably is made of a rubber material. A pressure plate 26 is provided for depressing the plunger assembly. A rod 28 is connected between the pressure plate 26 and seal 22.

Syringe 10 further includes a second moveable gasket or seal 30 located between the first seal 22 and the first end 18 of the tube. Seal 30 should be made of a resilient material, such as rubber, and may be identical to seal 22. A first chamber 32 is formed between the second seal 30 and the first end 18 of the tube. A second chamber 34 is formed between the second seal 30 and the first seal 22. A material, preferably a liquid medicine, is received in first chamber 32. A liquid such as a saline solution is received in second chamber 34.

As described above syringe 10 is in the fully loaded condition with the plunger not having been depressed. As best seen in FIG. 3 needle assembly 14 includes a first hollow needle 36 and a second hollow needle 38. Needle 36 could be a standard metal needle or a plastic tube, such as that which is used in a needleless system. A standard hollow needle support 40 connects the passage and hollow needle 36 to the passage and hollow needle 38 through a passage 42 inside needle support 40. Thus fluid on the inside of tube 12 may readily pass through hollow needle 38 needle, support 40 and out hollow needle 36 into an IV catheter port or otherwise into a medical patient. The outside surface of needle support may be threaded to match threads at first open end 18 of tube 12 or the needle support 40 may be otherwise attached to the open end 18 by means of glue or by some other means. Some or both of the outside surface of hollow needles 36 and 38 may also be threaded to match threads on the inside surface of the needle support 40 or one or both of the needles 36 and 38 may be glued or otherwise attached to the needle support 40.

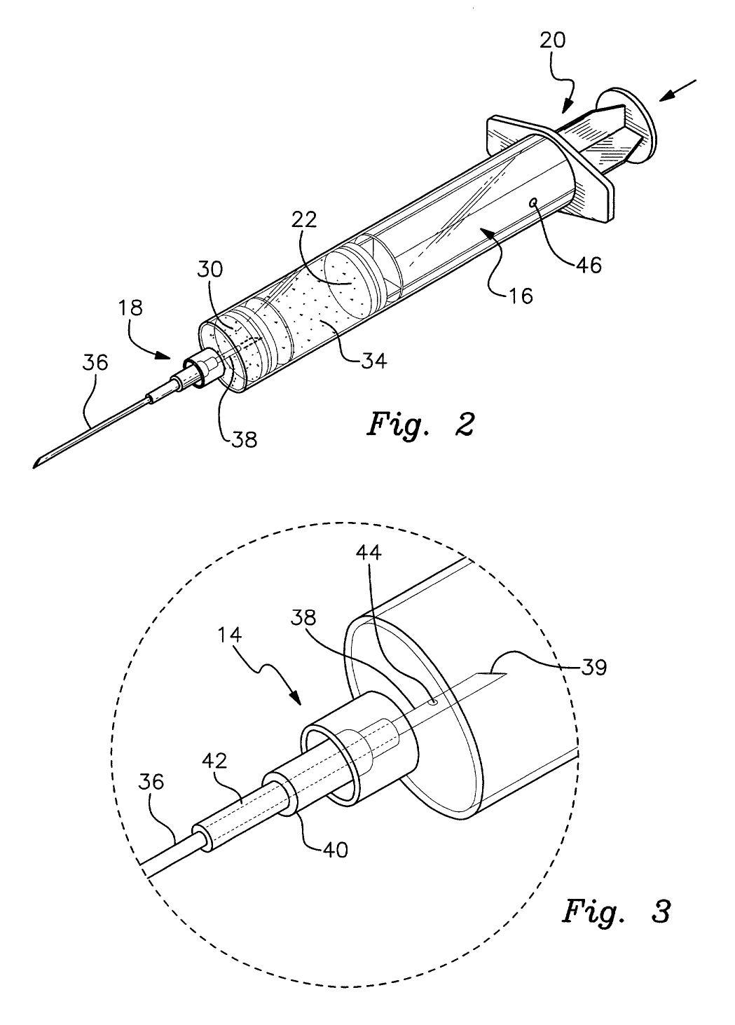

FIG. 2 shows plunger assembly 16 in a more fully depressed state. Since the fluid, such as saline solution, which is in second chamber 34, is a substantially incompressible liquid, depression of plunger 16 will cause first gasket 22 to exert force on the liquid 34 which will cause seal 30 to move toward the first open end 18 of tube 12. Movement of seal 30 towards first end 18 will place pressure on the liquid medicine within second chamber 32 causing the liquid medicine to be expelled through hollow needle 38 and out hollow needle 36. As seal 30 continues to travel toward open end 18 of the tube, hollow needle 38 will penetrate seal 30 and will extend into first chamber 34 which has been displaced toward open end 18 of the tube. Once hollow needle 38 has completely penetrated through seal 30 the liquid in chamber 34 will expel through hollow needle 38 and out hollow needle 36. If assembly 16 is depressed so far that needle 32 penetrates seal 30, which is also preferably made of a rubber material, it would be desirable to have a hole (not shown) in the hard plastic material behind seal 30 which aligns with needle 32 so that needle 32 will not be damaged.

In the situation where the medicine in chamber 32 is adenosine, the saline from chamber 34 quickly follows the adenosine into the IV tube thereby forcing the adenosine to enter the patient's general circulation in a very rapid manner without the need to use a second syringe to administer the saline. In addition the saline or other fluid from second chamber 34 will provide a clean IV tube.

In the embodiment as shown in FIG. 3 hollow needle 38 includes a side port 44 drilled therein. The side port 44 is provided so that once seal 30 has been initially penetrated the remaining medicine within first chamber 32 continues to be expelled until port 44 passes into seal 30, since the open end 39 of hollow needle 38 is for a time fully imbedded in seal 30 or has passed into chamber 34.

In order to easily fill the syringe during the filling process and to eliminate trapped air in second chamber 34 a hole or side port 46 may be drilled into the side of tube 12 near or just below second opening 20. Filling the first chamber 32 with liquid medicine and then inserting seal 30 behind it should not result in trapped air in first chamber 32 if done properly however, when the solution is placed in second chamber 34 it can become problematic to then place seal 30 with the plunger 16 behind it without trapping air in the second chamber 34. Side port 46 placed at the appropriate level in the tube would allow the plunger to be depressed into the tube (into the second chamber 34) and would allow trapped air or extra solution to be expelled. Once the first seal 22 reaches the side port 46 and passes it both chambers would be filled with medicine or solution and would be free of air pockets.

From the foregoing description of the preferred embodiment of the invention it will be apparent that many modifications may be made therein. It should be understood however, that this embodiment of the invention is an exemplification of the invention only and that the invention is not limited thereto. It is to be understood therefore that it is intended in the appended claims to cover all modifications as fall within the true spirit and scope of the invention.

What is claimed is:

1. A pre-loaded multi-chambered syringe comprising:
(a) an elongated hollow tubular barrel, said barrel having first and second open ends and having an inner wall;
(b) a needle assembly attached to said first end of said barrel;
(c) a plunger assembly slidably received inside said barrel through said second end of said barrel;
(d) said plunger assembly having a first seal;
(e) said first seal engaging said inner wall in substantially an interference fit;
(f) a second seal engaging said inner wall in a substantially interference fit;
(g) said second seal located inside said barrel between said first seal and said first end of said barrel wherein a first chamber is formed between said second seal and said first end of said barrel, and a second chamber is formed between said second seal and said first seal;
(h) said first chamber being substantially filled with a first material; said second chamber substantially filled with a liquid, wherein the movement of said first seal toward said first end of said barrel causes said second seal to slide toward said first end of said barrel thereby expelling said first material through said needle assembly;
(i) said second seal together with said inner wall maintaining a substantially liquid impervious seal between said first chamber and said second chamber;
(j) said impervious seal between said first and second chambers being maintained while said second gasket slides towards said first end of said barrel;
(k) said needle assembly adapted to penetrate said second seal so that an amount of liquid in said second chamber can be expelled through said needle assembly after the first material has been expelled.

2. A multiple chambered syringe comprising:
(a) an elongated hollow tube having an inside surface, and first and second open ends; said first end receiving a needle support; said second end receiving a plunger assembly; a portion of said plunger assembly forming a seal with said inside surface of said tube;
(b) moveable seal located between said plunger assembly and said first end;
(c) first chamber formed between said seal and said first end, and a second chamber formed between said seal and said plunger;
(d) a first hollow needle attached to needle support and extending into said first chamber;
(e) said first chamber being substantially filled with a first liquid;
(f) said second chamber being substantially filled with a second liquid, whereby when said plunger is depressed said seal will move toward said first opening for a first distance thereby expelling at least a portion of said first liquid through said needle, and when said plunger is further depressed, said seal will move toward said first opening a second distance wherein said needle will penetrate said seal expelling at least a portion of said second liquid through said needle.

3. A syringe as set forth in claim 2, further including a second hollow needle attached to said needle support and extending away from said tube.

4. A syringe as set forth in claim 2, wherein said seal is primarily made of rubber.

5. A syringe as set forth in claim 2, wherein said first hollow needle includes a side port located between the tip and the tip of said first needle and said needle support.

6. A syringe as set forth in claim 2, wherein the first liquid includes a medicine.

7. A syringe as set forth in claim 2, wherein said second liquid is saline solution.

8. A syringe as set forth in claim 3, wherein said second needle is adapted to be connected to an IV catheter port.

9. A syringe as set forth in claim 2, further including a side port in said tube located between said seal and said second opening in said tube.

10. A method for expelling at least two separate liquids from a multi-chambered syringe, said syringe including,
an elongated hollow tube having an inside surface, and first and second open ends; said first end receiving a needle support; said second end receiving a plunger assembly, a portion of said plunger assembly forming a seal with said inside surface of the tube; a moveable gasket located between said plunger and said first end of said tube; a first chamber formed between said gasket and said first end of said tube, and a second chamber formed between said gasket and said plunger;
a hollow needle attached to said needle support and extending into said first chamber; said first chamber being at least partially filled with a first liquid;
said second chamber being at least partially filled with a second liquid.
Said method comprising the steps of:
(a) depressing said plunger a first distance;
(b) moving said gasket towards first end of said tube;
(c) expelling an amount of said first liquid through said needle;
(d) depressing said plunger a second distance;
(e) penetrating said gasket with said needle and extending said needle into said second chamber and;
(f) expelling an amount of said second liquid through said needle.
